(19) United States
(12) Patent Application Publication
(10) Pub. No.: US 2009/0117088 A1
(43) Pub. Date: May 7, 2009

(54) CELLULAR INTERVENTION TO TREAT DAMAGED MYOCARDIUM

(75) Inventor: Pierre A. Grandjean, Warsage (BE)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/261,155

(22) Filed: Oct. 30, 2008

Related U.S. Application Data
(62) Division of application No. 11/216,295, filed on Aug. 31, 2005, now abandoned.

(51) Int. Cl. A61K 35/12; A61N 1/00

(52) U.S. Cl. 424/93.7; 607/3

(57) ABSTRACT

A system and method for treating damage myocardial tissue includes delivering replacement cells to the myocardium of a patient and electrically stimulating the spinal column of the patient to affect a cellular environment within the myocardium.

### CROSS REFERENCE TO RELATED APPLICATIONS

The present patent disclosure relates to and incorporates by reference co-pending U.S. patent application Ser. No. 11/216,295 filed Aug. 31, 2005, entitled, “Cellular Intervention to Treat Damaged Myocardium, U.S. patent application Ser. No. 09/999,723 filed Oct. 26, 2001, entitled, “Method and Apparatus to Minimize the Effects of a Cardiac Insult,” now issued as U.S. Pat. No. 7,010,345, and co-pending U.S. patent application Ser. No. 10/754,173 filed Jan. 8, 2004 and entitled, “Medical Fluid Delivery System”, now issued as U.S. Pat. No. 7,274,966.

### BACKGROUND OF THE INVENTION

The present invention relates generally to systems and methods for treating coronary heart disease. In particular, the present invention relates to repopulating and/or regenerating damaged or diseased myocardial tissue with introduced replacement cells.

Coronary Artery Disease (CAD) is a major health problem worldwide. In persons having CAD, the formation of plaque narrows the coronary artery and reduces the supply of oxygen and nutrients to the heart, which can cause acute myocardial infarction (AMI). AMI is a condition of irreversible necrosis of the heart muscle that results from prolonged ischemia. Over time, the damaged or diseased regions of the myocardium associated with AMI are replaced with scar tissue, which decreases the contraction of the heart and can create electrical abnormalities. As a result, survivors of AMI have an increased risk of developing heart failure.

Current treatments for AMI survivors focus on pharmacological and surgical approaches that are designed to achieve reperfusion and minimize ventricular damage. These therapies, however, do not address myocardial necrosis and its effects on heart function. Cellular replacement techniques to address myocardial necrosis and/or myocardial depressed contractility (akynesia) are under clinical investigation. These techniques entail supplying replacement cells to repair or enhance damaged or diseased portions of the myocardium. Preliminary results suggest that this form of therapy may positively impact the functioning of the heart.

These conventional cellular replacement techniques, however, have yielded low survival rates for the introduced replacement cells, as well as poor engraftment, when introduced into the myocardial tissue of patients. Cell survival rates are poor, with typically about 20 percent of the replacement cells surviving 1 week after being delivered to the myocardium. One report indicates survival rates for the replacement cells of less than 1 percent. See Taylor, Int'l Journal of Cardiology, 95 Suppl. 1 (2004): S13-S15. While the precise reasons for the low survival rates are not known, they may be associated with a lack of necessary nutrients and gaseous exchange.

Thus, among other things a need exists for improved apparatus and methods for addressing myocardial necrosis for survivors of AMI and other subjects having scars or lesions that interfere with conduction and/or mechanical electrical contraction of the heart.

### BRIEF SUMMARY OF THE INVENTION

The present invention treats damaged myocardial tissue in a patient with a combination of replacement cells delivered to the myocardium and electrical stimulation applied to the spinal column. The electrical stimulation affects a cellular environment for the replacement of cells within the myocardium.

### BRIEF DESCRIPTION OF THE DRAWINGS

FIG. 1 is a schematic representation of a nerve stimulator system applying electrical stimulation to the spine of a patient,

FIG. 2 is a schematic representation of a cell delivery device delivering replacement cells to a damaged myocardial tissue region of the patient of FIG. 1

FIG. 3 is a cross-sectional view of the spinal column of the patient of FIG. 1 showing placement of a stimulation electrode within the epidural space.

### DETAILED DESCRIPTION

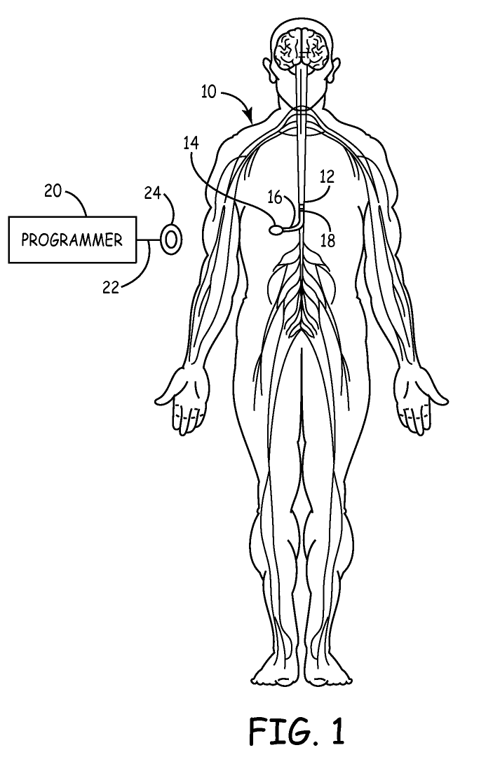

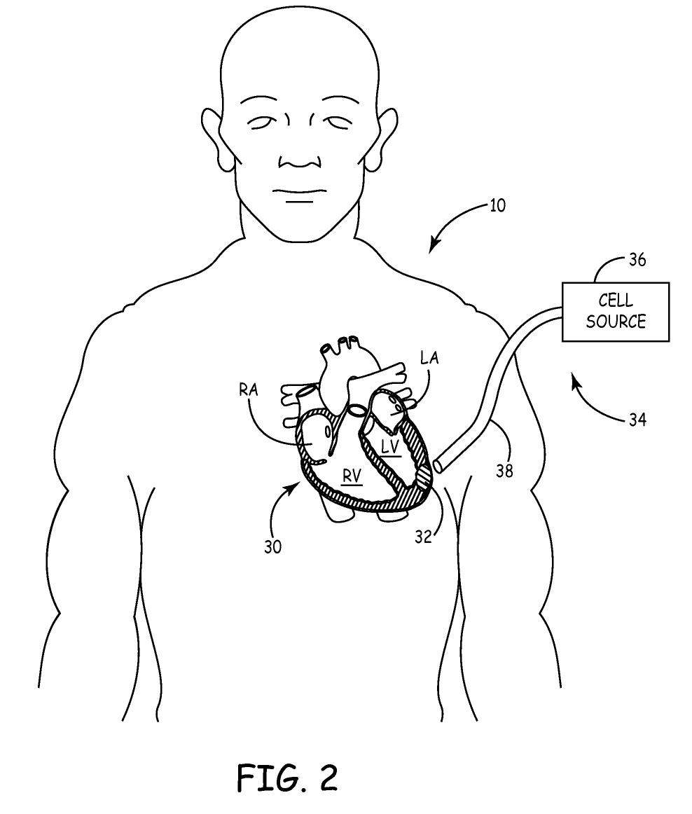

FIGS. 1 and 2 illustrate a method and system for treating a patient 10 having AMI with a combination of replacement cells delivered to the myocardium and electrical stimulation applied to the spinal column. The present invention combines cell-replacement with electrical stimulation of the spinal column of a patient to affect the cellular environment of the myocardium and form a more hospitable cellular environment for replacement cells. The beneficial effects of modifying the cellular environment may include, for example, enhanced cell survival of replacement cells, enhanced host myocardial tissue, enhanced ability of replacement cells to differentiate into more suitable cell types, and/or enhanced ability of replacement cells to bond (or engraft) with host myocardial tissue or structural matrixes.

The more hospitable cellular environment may result from improvements in myocardial blood flow caused by spinal cord stimulation (SCS). SCS has been employed to treat various conditions, including angina pectoris, which is a symptom of myocardial ischemia. SCS has been demonstrated to improve myocardial blood flow at the microvascular level in angina patients using positron emission tomography (PET), with improvement in regional blood flow to ischemic areas. See Latif, et al., Clinical Cardiology 24: 533-541 and Hautvast, et al., The American Journal of Cardiology 77 (1996): 462 -467. For further discussion regarding the effects of SCS on myocardial blood flow, see also Jessurun, et al., European Journal of Pain 7 (2003): 507-512. SCS has been documented to have numerous positive effects on patients suffering from angina pectoris, including both antianginal and anti-ischematic effects. See Aronow, et al., Current Treatment Options in Cardiovascular Medicine 6:79-83. Improved blood flow as a result of SCS may play a key role in mediating these positive effects in angina pectoris patients.

FIG. 1 is a schematic view of patient 10 that illustrates electrical stimulation being applied to spine 12. Nerve stimulator 14 supplies electrical stimulation pulses via lead 16 to electrode 18. These electrical stimulation pulses are applied to spine 12 by electrode 18, which is located within, or adjacent to, spine 12.

Nerve stimulator 14 may be programmed to provide a predetermined stimulation dosage in terms of pulse amplitude, pulse width, pulse frequency, or duty cycle. Programmer 20, in conjunction with conductor 22 and antenna 24, may be used to provide stimulation parameters to nerve stimulator 14 via telemetry. This permits attending medical personnel to provide stimulation parameters to nerve stimulator 14 after implantation using radio frequency communication.

Nerve stimulator 14 may be implanted in the abdomen or any other portion of the body of patient 10. In some embodiments, nerve stimulator 14 is located outside patient 10. Nerve stimulator 14 can be an implantable pulse generator with one or more implanted leads 16, a partially implantable nerve stimulation system including an external transmitter and an implantable receiver powered by the transmitter, or an external stimulation system having an external pulse generator and leads. Examples of suitable nerve stimulators 14 include, but are not limited to, Model 7425 Itrel® 3 neurostimulators, Model 7427 Synergy® neurostimlators, Model 7479 SynergyPlus® neurostimulators, Model 7479B SynergyCompact® neurostimulators, and 3271 Mattrix® receivers—all of which are commercially available from Medtronic, Inc.

Any type of lead 16 with any number and configuration of electrodes 18 may be used to apply electrical stimulation to the spinal column. Examples of suitable leads include percutaneous leads, surgical leads, and transcutaneous leads.

FIG. 2 is a schematic view illustrating the delivery of replacement cells to heart 30 of patient 10. Heart 30 has a right ventricle RV, a left ventricle LV, a right atrium RA, and a left atrium LA. In the example of FIG. 2, left ventricle LV has a damaged myocardial tissue region 32. Cell delivery device 34 (which includes replacement cell source 36 and delivery conduit 38) delivers replacement cells to damaged myocardial tissue region 32. To affect the cellular environment for the replacement cells in myocardial tissue region 32, nerve stimulator 14 applies electrical stimulation to spine 12. The timing of the electrical stimulation with respect to the delivery of replacement cells is selected to produce the cellular environment that is more hospitable for the replacement cells.

Cell delivery device 34 can take various forms and use various methods to deliver replacement cells to damaged myocardial tissue region 32. Such cell delivery devices and methods are well known in the art. See, for example, U.S. Pat. No. 6,805,860 and pending U.S. Pat. App. 2004/0158289 (Application Ser. No. 722,115) by Girouard, et al. As shown in FIG. 2, cell delivery device 34 is a catheter-based delivery system in which replacement cell source 36 comprises a pump or syringe and delivery conduit 38 comprises a catheter. In other embodiments, replacement cell source 36 comprises a syringe and delivery conduit 38 comprises a needle.

The replacement cells may be delivered to the myocardium via any route, including intravenously, transvenuously, intramyocardially, or other routes known in the art. After being located in close proximity to heart 30, the replacement cells can be delivered to the myocardium by either injecting cells directly into the myocardium or by introducing the replacement cells into a vessel supplying blood to the myocardium.

To inject cells into the myocardium, delivery conduit 38 may be positioned, for example, in or adjacent to the left ventricle, the right ventricle, the left atrium, or the right atrium. In some embodiments, the replacement cells are injected near, and/or into, an infarcted region of the myocardium (e.g., myocardial region 32) or other damaged or diseased region of the myocardium. The replacement cells may be injected into the myocardium using a single injection or a plurality of injections. In some embodiments, injections may be separated from each other in time by hours, days, weeks, months, or years.

Various methods may be employed to locate damaged myocardial tissue regions 32. For example, electrophysiology (e.g., electrocardiograms) or any other locating methods known in the art may be used to locate damaged myocardial tissue. See U.S. Pat. App. 2004/0158289 (Application Ser. No. 722,115) by Girouard, et al.

The application of the electrical stimulation by nerve stimulator 14 has a temporal relationship to the delivery of the replacement cells to the myocardium. For example, the electrical stimulation may be applied prior to delivery of the replacement cells, during delivery of the replacement cells, after delivery of the replacement cells, or any combination of these. The timing of the delivery of the replacement cells to the myocardium by cell delivery device 34 may be determined as a function of the timing of the application of electrical stimulation to the spinal column by nerve stimulator 14.

In one embodiment, nerve stimulator 14 is initiated at a given time (e.g., about 1 month) prior to delivery of replacement cells to the myocardium. SCS delivered by nerve stimulator 14 may be continued and/or modified after delivery of the replacement cells by cell delivery device 34. In some embodiments, SCS is continued for a set period of time after delivery of the replacement cells (e.g., about 1 month), while in other embodiments SCS is continued indefinitely.

The nature of the electrical stimulation applied to the spinal column by nerve stimulator 14 may vary. The electrical stimulation can be applied to the spinal column using any SCS stimulus parameters known in the art, such as amplitude, pulse width, and pulse rate. In one embodiment, the stimulation parameters include a pulse amplitude of about 5 volts (V), a pulse width between about 10 microseconds and about 1,000 microseconds, and a pulse rate between about 30 Hertz (Hz) and about 80 Hz. The electrical stimulation can be applied in a continuous mode (i.e., continuous stimulation), a cycling mode (i.e., on for a set period of time and off for a set period of time), pursuant to any other mode known in the art, or in any combination of these.

In some embodiments, the nature of the electrical stimulation applied to the spinal column may be varied by nerve stimulator 14 during the course of treatment as a function of various factors. For example, in one embodiment, a first stimulation protocol is used by nerve stimulator 14 before delivery of the replacement cells to the myocardium, a second stimulation protocol is used during delivery of the replacement cells to the myocardium by cell delivery device 34, and a third stimulation protocol is used after delivery of the replacement cells to the myocardium.

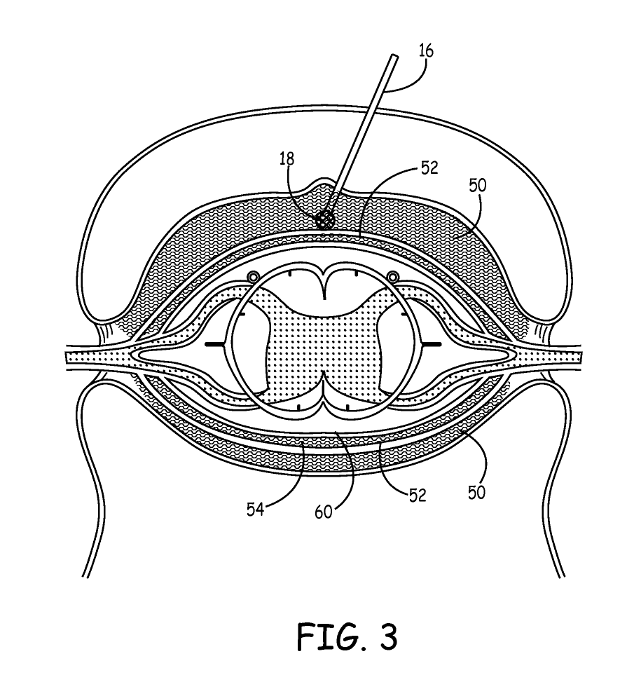

In some embodiments, lead(s) 16 are placed in the epidural space of the spinal column so that one or more electrodes 18 are located close enough to the dorsal horn to stimulate specific large nerve fibers. FIG. 3 shows a cross sectional view of spine 12 and adjacent tissue of patient 10 of FIGS. 1 and 2, with lead 16 and electrode 18 implanted in epidural space 50. Subdural space 54 located between dura mater 52 and arachnoid membrane 60 are included in FIG. 3 purposes of orientation.

Lead 16 and electrode 18 can be placed in epidural space 50 either surgically or percutaneously through a needle (e.g., a Tuohy needle). Optimal lead placement sites for angina pectoris patients include upper thoracic or lower cervical spinal locations. See U.S. Pat. No. 5,085,584. Thus, in some embodiments, lead 16 is positioned so that one or more electrodes are located in epidural space 50 of the upper thoracic or lower cervical vertebrae. In one embodiment, electrode(s) 18 is located in the epidural space of thoracic vertebrae T1, T2, and/or T3.

In other embodiments, SCS is accomplished by nerve stimulator 14 through transcutaneous electrical nerve stimulation (TENS). One or more electrodes are placed on skin that overlies, or is near to, the spinal column, and electrical stimulation is applied from the electrodes to the spinal column through the skin.

Any variety or combination of suitable replacement cells known in the art may be utilized in conjunction with the present invention. In some embodiments, the replacement cells are autologous to help avoid host rejection. In other embodiments, the replacement cells may be allogenic, xenogenic, or a combination of any of these (including autologous). While a risk of cell rejection is associated with allogenic and xenogenic cells, the use of such cells, when taken from established cell lines, overcomes the need to harvest and expand cells. Allogenic and xenogenic cells may be treated to reduce the risk of rejection.

Other examples of cell types useful in this invention include, but are not limited to, stem cells and progenitor cells derived from bone marrow and from blood; skeletal muscle progenitor cells (skeletal muscle myoblasts or adult stem cells derived from skeletal muscle are synonyms); cardiac progenitor cells (c-kit +); other stem cells; satellite cells; other cells; and any combination of these in any proportion. For further discussion of replacement cells, see, for example, U.S. Pat. No. 6,671,558, U.S. Pat. No. 6,805,860, and U.S. Pat. App. 2004/0158289 (Application Ser. No. 722,115) by Girouard, et al.

In some embodiments, cells are harvested from a patient and cultured outside the patient for a period of days or weeks to produce a population of replacement cells for delivery to the myocardium of the patient. In one embodiment, one or more electrodes 18 are implanted into, or near, the spinal column of the patient during the same procedure in which the cells are harvested from the patient.

A variety of exogenous stimuli may be applied to the replacement cells being delivered to the myocardium. For instance, the replacement cells may be conditioned in vitro with mechanical stimuli (e.g., applying cyclical mechanical stress to replacement cells to simulate the cyclical contraction of cardiac muscle cells in vivo), electrical stimuli (e.g., subjecting replacement cells to electrical conditions that simulate the electrical conditions in the myocardium which result in contraction of the heart muscle), or biological stimuli (e.g., exposure to differentiation factors, growth factors, angiogenic proteins, survival factors, and/or cytokines). See, for example, U.S. Pat. App. 2004/0158289 (Application Ser. No. 722,115) by Girouard, et al. The conditioning may include continuous or intermittent exposure to the exogenous stimuli.

In some embodiments, the replacement cells may also be transfected ex vivo or in vitro to express one or more desired proteins (e.g., connexins and/or sodium channel subunits and/or calcium channel subunits).

The replacement cells can be coated or otherwise incorporated into a carrier to, for example, assist in localizing the replacement cells in the myocardium. In some embodiments, the replacement cells are delivered to the myocardium in a polymeric matrix made up of one or more synthetic or natural polymers compatible with the replacement cells. See, for example, U.S. Pat. No. 6,671,558. In one embodiment, the polymeric matrix can be in the form of a porous scaffold, whereby the polymer matrix is seeded with replacement cells. See U.S. Pat. No. 6,671,558.

In some embodiments, one or more growth factors or other chemical or biological agents may be injected into the myocardium before, after, and/or simultaneous with delivery of the replacement cells to help provide a more optimal environment for survival, engraftment, and/or differentiation of the replacement cells.

Thus, as described above, the present invention includes a system and method for delivering replacement cells to myocardial tissue of a patient and electrically stimulating the spinal column of the patient to affect a cellular environment for the replacement cells within the myocardium.

Although the present invention has been described with reference to preferred embodiments, workers skilled in the art will recognize that changes may be made in form and detail without departing from the metes and bounds and scope of the invention.

1. A method for treating a patient's damaged myocardial tissue, the method comprising:
preparing cells capable of enhancing myocardial tissue;
delivering the cells to the damaged myocardial tissue; and
for at least a day prior to delivering the cells, electrically stimulating the Patient's spinal column to affect a cellular environment within the patient's myocardium.

2. A method according to claim 1, wherein at least a portion of the electrical stimulation is applied for at least about a month prior to delivering the cells.

3. A method according to claim 1, wherein preparing the cells comprises harvesting the cells from the patient and culturing the cells outside the patient.

4. A method according to claim 1, and further comprising:
delivering a growth factor to the damaged myocardial tissue.

5. A method according to claim 1, wherein the cells delivered to the damaged myocardial tissue are dispersed in a carrier material.

6. A method for treating a patient's myocardium, the method comprising:
delivering cells to the patient's myocardium; and
for at least a day prior to delivering the cells, applying, electrical stimulation capable of affecting myocardial blood flow to the patient's spinal column.

7. A method according to claim 6, wherein applying the electrical stimulation begins about one month prior to delivering the cells.

8. The method of claim 7 comprising terminating application of the electrical stimulation at least a about a month following delivering the cells.

9. The method of claim 7 comprising suspending delivery of the electrical stimulation while delivering the cells.

10. The method of claim 7 wherein the electrical stimulation is applied by an electrode located within the spine.

11. The method of claim 7 wherein the electrical stimulation is applied by an electrode located adjacent the spine.

12. The method of claim 7 further comprising varying parameters of the electrical stimulation.

13. The method of claim 6 comprising terminating application of the electrical stimulation at least a day following delivering the cells.

14. The method of claim 6 comprising suspending delivery of the electrical stimulation while delivering the cells.

15. The method of claim 6 wherein the electrical stimulation is applied by an electrode located within the spine.

16. The method of claim 6 wherein the electrical stimulation is applied by an electrode located adjacent the spine.

17. The method of claim 6 further comprising varying parameters of the electrical stimulation.

18. The method of claim 1 comprising suspending delivery of the electrical stimulation while delivering the cells.

19. The method of claim 1 wherein the electrical stimulation is applied by an electrode located within the spine.

20. The method of claim 1 wherein the electrical stimulation is applied by an electrode located adjacent the spine.

21. The method of claim 1 further comprising varying parameters of the electrical stimulation.
